(19) United States
(12) Patent Application Publication
(10) Pub. No.: US 2023/0302003 A1
(43) Pub. Date: Sep. 28, 2023

(54) COMPOSITIONS AND METHODS FOR TREATING A CORONAVIRUS INFECTION

(71) Applicant: Antirna Incorporated, Burlington, MA (US)

(72) Inventor: Suhaib Siddiqi, Burlington, MA (US)

(21) Appl. No.: 18/019,940

(22) PCT Filed: Aug. 6, 2021
(86) PCT No.: PCT/US21/44969

§ 371 Date: Feb. 6, 2023

Related U.S. Application Data
(60) Provisional application No. 63/062,093, filed on Aug. 6, 2020.

(51) Int. Cl. A61K 31/52; A61P 31/14

(52) U.S. Cl. CPC A61K 31/52; A61P 31/14

(57) ABSTRACT

The application relates to methods for treating a viral infection, such as COVID-19 infection, comprising administering to the subject an inhibitor of S-adenosylhomocysteine (SAH) hydrolase.

### RELATED APPLICATIONS

This application claims the benefit of priority to U.S. Provisional Patent Application Ser. No. 63/062,093, filed Aug. 6, 2020, the contents of which are hereby incorporated herein by reference in their entirety.

### BACKGROUND

Coronavirus disease 2019 (COVID-19) is an infectious disease caused by severe acute respiratory syndrome coronavirus 2 (SARS-CoV-2). Common symptoms include fever, cough, fatigue, shortness of breath, and loss of smell and taste. While the majority of cases result in mild symptoms, some progress to acute respiratory distress syndrome (ARDS) possibly precipitated by cytokine storm, multi-organ failure, septic shock, and blood clots. There are no approved vaccines nor specific antiviral treatments for COVID-19. Based on accumulated data, interferons, ribavirin, remdesivir, chloroquine and hydroquinone, favipiravir, ritonavir, lopinavir, and other broad-spectrum antiviral drugs could potentially be used to treat COVID-19, by inhibiting the viral RNA replication, and viral protein expression. However, none of these drugs have proved to be effective in treating COVID-19.

Though there are treatments, such as dexamethasone, that can alleviate certain symptoms, such as difficulty breathing, these treatments have failed to adequately suppress the viral infection. Disease management thus typically involves the treatment of symptoms, supportive care, isolation, and experimental measures. Hence, there is an urgent need to find new strategies for treating COVID-19.

### SUMMARY

In some aspects, methods of treating coronavirus infection in a subject are disclosed. Such methods include administering to the subject a compound of Formula I or Formula II, or a pharmaceutically acceptable salt thereof. Numerous embodiments are further provided that can be applied to any aspect of the present invention described herein. For example, in some embodiments, the compound is formulated for oral, rectal, intravenous, or subcutaneous formulation. In some embodiments, the compound is formulated for inhalation. The compound may be formulated as a dry powder. In some embodiments, the coronavirus is selected from SARS-CoV, MERS-CoV, HCoV, HKU1, and SARS-CoV-2. In some embodiments, the subject has SARS-CoV-2 infection, which has been confirmed by reverse-transcription polymerase chain reaction (RT-PCR) from respiratory tract or blood specimens.

In some aspects, methods of treating coronavirus infection in a subject are disclosed. Such methods include administering to the subject an inhibitor of S-adenosylhomocysteine (SAH) hydrolase, such as a compound selected from Table 1. Numerous embodiments are further provided that can be applied to any aspect of the present invention described herein. For example, in some embodiments, the inhibitor of SAH hydrolase is formulated for oral, rectal, intravenous, or subcutaneous formulation. In some embodiments, the inhibitor of SAH hydrolase is formulated for inhalation. The inhibitor of SAH hydrolase may be a dry powder for inhalation. In some embodiments, the coronavirus is selected from SARS-CoV, MERS-CoV, HCoV, HKU1, and SARS-CoV-2. In some embodiments, the subject has SARS-CoV-2 infection, such as an infection confirmed by reverse-transcription polymerase chain reaction (RT-PCR) from respiratory tract or blood specimens.

In some aspects, pharmaceutical compositions comprising a compound of Formula I or Formula II or a pharmaceutically acceptable salt thereof are disclosed. Numerous embodiments are further provided that can be applied to any aspect of the present invention described herein. For example, in some embodiments, the composition is formulated for oral, rectal, intravenous, or subcutaneous formulation. In some embodiments, the composition is formulated for inhalation. The composition may be formulated as a dry powder.

In some aspects, inhaler compositions comprising the pharmaceutical composition described herein are disclosed.

In some aspects, inhalers comprising the pharmaceutical composition or the inhaler composition described herein are disclosed.

### DETAILED DESCRIPTION

The RNA-dependent RNA polymerase (RdRp) of coronaviruses is a well-established drug target. The active site of the RdRp is highly conserved among positive-sense RNA viruses (te Velthuis 2014). These RdRps have low fidelity (Selisko et al. 2018), allowing them to recognize a variety of modified nucleotide analogues as substrates. Such nucleotide analogues may inhibit further RNA-polymerase catalyzed RNA replication making them candidates as anti-viral agents (McKenna et al. 1989; Öberg 2006; Eltahla et al. 2015; De Clercq and Li 2016). RdRps in SARS-CoV and SARS-CoV-2 have nearly identical sequences (Elfiky 2020). Recently, the SARS-CoV-2 RdRp was cloned (Chien et al. 2020) and the RNA polymerase complex structure was determined (Gao et al. 2020), which will help guide the design and study of RdRp inhibitors. Many of the agents listed above are directed towards RdRp.

Inhibitors of S-adenosylhomocysteine (SAH) hydrolase can be used to treat COVID-19 infection. The SAH hydrolase inhibitors are potent antiviral agents. SAH hydrolase plays a key role in methylation reactions depending on S-adenosylmethionine (AdoMet) as a methyl donor. SAH hydrolase inhibitors have been shown to exert broad-spectrum antiviral activity against pox-, paramyxo-, rhabdo-, filo-, bunya-, arena-, and reoviruses. They also interfere with the replication of human immunodeficiency virus through inhibition of the Tat transactivation process.

S-Adenosylhomocysteine, formed after the donation of the methyl group of S-adenosylmethionine to a methyl acceptor, is hydrolyzed to adenosine and homocysteine by SAH hydrolase physiologically. The administration of the inhibitors of SAH hydrolase to cells or animals normally results in an accumulation of cellular S-adenosylhomocysteine higher than those found in controls, which is often accompanied by a simultaneous rise in S-adenosylmethionine because of the feedback inhibition by S-adenosylhomocysteine on most methylation reactions. SAH hydrolase has become a tantalizing pharmacological target for inhibition since its blockade can affect cellular methylation of phospholipids, proteins, small molecules, DNA, and RNA. Indeed, all of these different methylation reactions have been found to be inhibitable by the nucleoside inhibitors/substrates of S-adenosylhomocysteine hydrolase. Among the interesting effects are the activation of genes, induction of cellular differentiation, increased expression of transcription factors, and sometimes the repression of genes. Furthermore, some of the nucleosides show remarkable antiviral activities in vitro and in vivo. However, the mode of action of the inhibitors appears complex. Although the inhibition of methylation might account for some of the biological effects, the ability of some of the nucleoside inhibitors to undergo metabolic phosphorylation to nucleotides may account for part of their biological activities.

SAH hydrolase catalyzes the reversible hydrolysis of SAH to adenosine and homocysteine through an oxidation/reduction reaction involving the cofactor NAD. Inhibition of SAH hydrolase results in the accumulation of SAH, which causes a product inhibition of S-adenosyl-L-methionine-dependent methyltransferases playing an essential role in the methylation of lipids, proteins, and nucleic acids. Therefore, SAH hydrolase has been investigated for the development of anti-viral agents.

The range of antiviral activity of SAH hydrolase inhibitors is very wide and diverse. These compounds are effective against particular DNA-containing viruses, such as vaccinia virus (VV) and human cytomegalovirus (HCMV). Many (−)-RNA viruses [parainfluenza, measles, Ebola, stomatitis and Varicella±Zoster (VZV) viruses], double-stranded RNA viruses (rheo- and rotaviruses) and retroviruses (human immunodeficiency viruses types 1 and 2, HIV-1 and HIV-2) belonging to (+)-RNA viruses are also sensitive to these inhibitors.

SAH hydrolase inhibitors are described herein as antiviral agents for treating COVID-19 infection. Accordingly, in certain embodiments, the invention provides methods of treating COVID-19 infection in a subject by administering to the subject an inhibitor of SAH hydrolase.

Non-limiting examples of Inhibitors of SAH hydrolase are shown in Table 1 below.

TABLE 1
Non-limiting examples of inhibitors of
S-adenosylhomocysteine (SAH) hydrolase.

| Target Protein | Compound name (if any) | Systematic name |
|---|---|---|
| SAH hydrolase | 3-Deazaneplanocin A | (1S,2R,5R)-5-(4- |
| | (DZNep) | aminoimidazo[4,5- |
| | | c]pyridin-1-yl)-3- |
| | | (hydroxymethyl)cyclopent- |
| | | 3-ene-1,2-diol |
| SAH hydrolase | D-eritadenine | (2R,3R)-4-(6-Aminopurin- |
| | | 9-yl)-2,3-dihydroxybutanoic |
| | | acid |
| SAH hydrolase | (S)-DHPA | (S)-9-(2,3- |
| | | dihydroxypropyl)adenine |
| SAH hydrolase | adenosine dialdehyde | |
| | (ADA) | |
| SAH hydrolase | (−)5′-Nor-aristeromycin | |

Previously synthesized (−)5′-Nor-aristeromycin (Siddiqi et al., J. Med. Chem. 1994, 37 551) showed potent broad-spectrum antiviral activity against a variety of (−) and (+)-RNA viruses, through SAH hydrolase inhibition. (−)5′-Nor-aristeromycin showed activity against a variety of viruses, significantly toward cytomegalovirus (CMV), Vaccinia virus (VV), vesicular stomatitis virus (VSV), parainfluenza-3 virus and reovirus-1 (MIC50 values ranging from 0.07 to 7 mg/mL) (S. Siddiqi et al J. Med. Chem 1994, 4, 551-554.)

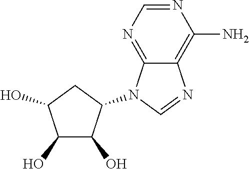 [SMILES: Nc1ncnc2c1ncn2[C@H]1C[C@@H](O)[C@H](O)[C@@H]1O]

### (−)5′-Nor-aristeromycin (a Carbocyclic Nucleoside)

Of particular interest is the broad-spectrum potent activity of (−)-5′-aristeromycin, which is attributed to its inhibitory effects of (S)-adenosyl-L-homocysteine (AdoHcy), and virus replication (S. Siddiqi et al J. Med. Chem 1994, 4, 551-554.). Therefore, (−)-5′-aristeromycin and it is derivatives and analogs are expected to be anti-COVID-19. (−)-5′-aristeromycin and its derivatives/analogs prevent COVID-19 virus replication, thus beneficial for the treatment of COVID-19 (Coronavirus) infected patients.

The (−)5′-nor-aristeromycin (a carbocyclic nucleoside), a SAH Hydrolase inhibitor, and its derivates are antiviral agents against COVID-19. The SAH hydrolase inhibitors have not been investigated as antiviral agents for COVID-19. (−)5′-Nor-aristeromycin, a SAH hydrolase inhibitor interferes with S-adenosylmethionine (SAM)-dependent methylation reactions, particularly those involved in the ‘capping’ of viral mRNA. (−)5′-Nor-aristeromycin and its derivatives may logically be expected to be effective in the treatment of COVID-19 virus infections.

(−)-5′-Noraristeromycin enantiomers were evaluated for antiviral activity against a large number of viruses and found to display an antiviral activity spectrum characteristic of (S)-adenosyl-L-homocysteine hydrolase inhibitors. It exhibited potent antiviral activity against a variety of viruses including RNA virus.

2,6-Chloro-5′-nor-purine derivative (2) is also synthesized. (See S. Siddiqi, et. al. J Med Chem. 1995 Mar. 31; 38(7): 1174-1188)

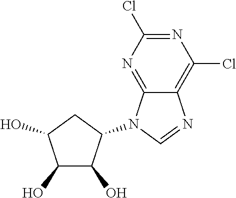 [SMILES: O[C@@H]1[C@H](O)[C@@H](n2cnc3c(Cl)nc(Cl)nc32)C[C@H]1O]

The chloro substituents at the 2 and 6 position provide reactive sites to attach various substituents for the purpose of exploring structure activity relationships (SAR) and optimize activity.

In one aspect, disclosed herein is a method of treating a coronavirus infection and/or associated inflammation, comprising administering to a subject in need thereof an inhibitor of SAH hydrolase (e.g., a compound of Formula I or Formula II). In certain aspects, also described herein is a pharmaceutical composition comprising an inhibitor of SAH hydrolase, e.g., a compound of Formula I or Formula II.

### Exemplary Inhibitors of SAH Hydrolase

In some embodiments, disclosed herein is a compound of Formula I or Formula II.

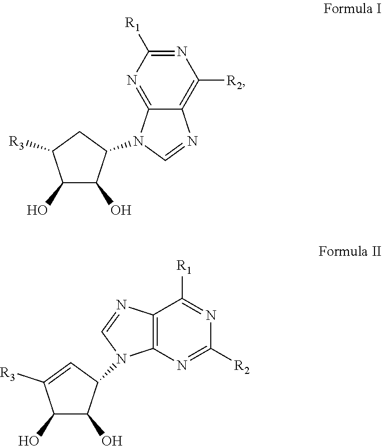

or a pharmaceutically acceptable salt thereof;
wherein
R₁ is H, halogen, NH₂, OH, SH, NH-Allyl, NH-alkyl, NH-aryl, NH-alkynyl, NH—CH₂—CH₂—O—CH₂—CH₂—O—CH₃, NH-PEG, O-alkyl, O-aryl, O-alkynyl, O-PEG, S-alkyl, S-aryl, S-alkynyl, S-PEG, alkyl, alkynyl, aryl, heteroaryl, or heterocyclyl;
R₂ is selected from H, halogen, NH₂, OH, SH, NH-Allyl, NH-alkyl, NH-aryl, NH-alkynyl, NH—CH₂—CH₂—O—CH₂—CH₂—O—CH₃, NH-PEG, O-alkyl, O-aryl, O-alkynyl, O-PEG, S-alkyl, S-aryl, S-alkynyl, S-PEG, alkyl, alkynyl, aryl, heteroaryl, and heterocyclyl; and
R₃ is selected from —H, —OH, -alkyl-OH, -alkylene-OH, alkyl, alkynyl, aryl, heteroaryl, and heterocyclyl.

In some embodiments, R₁ is H. In some embodiments, R₁ is a halogen, such as Cl. In some embodiments, R₂ is H. In some embodiments, R₂ is a halogen, such as Cl. In some embodiments, R₂ is NH₂. In some embodiments, R₃ is —H. In some embodiments, R₃ is —OH. In some embodiments, R₃ is, -alkyl-OH, such as methyl-OH. In some embodiments, R₃ is alkyl, such as methyl.

In certain embodiments, the compound of Formula I is selected from:

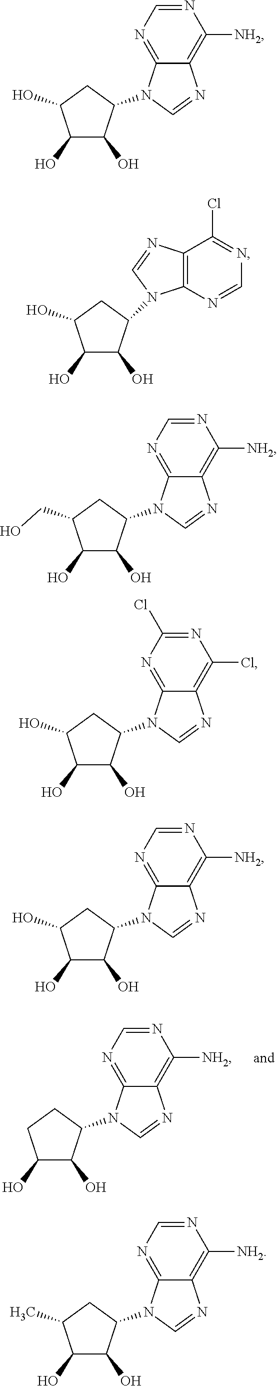

In certain embodiments, the compound of Formula II is selected from:

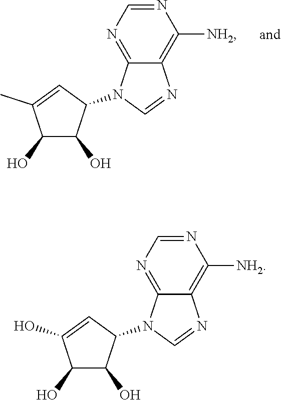

In some embodiments, the purine in Formula I or Formula II is replaced with a pyrimidine. In some embodiments, the purine in Formula I or Formula II is a guanosine.

CoVs are positive-stranded RNA viruses with a crown-like appearance under an electron microscope (coronam is the Latin term for crown) due to the presence of spike glycoproteins on the envelope. Based upon the known broad-spectrum anti-viral activity of (−)-5′-noraristeromycin, as a nontoxic antiviral agent, and its action through SAH hydrolase inhibition, it is expected to show activity against the SARS-CoV viruses including CoV-2 virus, which are positive-stranded RNA virus.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a viral infection, is well understood in the art, and includes administration of a composition which reduces the frequency or severity of, or delays the onset of, symptoms of a medical condition in a subject relative to a subject which does not receive the composition. Thus, prevention of coronavirus infection includes, for example, reducing the incidence of coronavirus infection in a population of patients receiving a prophylactic treatment relative to an untreated control population, and/or delaying the incidence and/or reducing the severity of coronavirus infection in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skill in the art to result in chemically stable compounds which can be readily synthesized, e.g., by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen atoms in a given structure with a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen atoms in a given structure with the substituents mentioned above. More preferably, one to three hydrogen atoms are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto.

Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkoxycarbonylalkyl” refers to a group

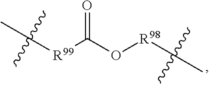

wherein R⁹⁸ and R⁹⁹ are both alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

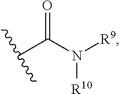

wherein R⁹ and R¹⁰ each independently represent a hydrogen or hydrocarbyl group, or R⁹ and R₁₀ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

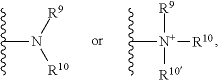

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group. The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

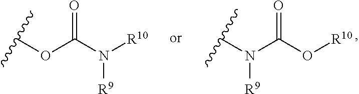

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

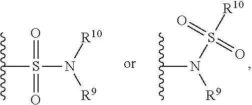

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

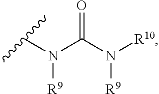

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

As used herein, the term “coronavirus” refers to a virus belonging to the subfamily Orthocoronavirinae, in the family Coronaviridae, order Nidovirales, and realm Riboviria. A coronavirus is an enveloped virus with a positive-sense single-stranded RNA genome and a nucleocapsid of helical symmetry. Coronaviruses include, for example, the following human viruses: human coronavirus 229E, human coronavirus OC43, SARS-CoV, HCoV, HKU1, MERS-CoV, and SARS-CoV-2. In some embodiments, coronavirus is SARS-CoV-2.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

### Pharmaceutical Compositions

The compositions and methods of the present invention may be utilized to treat a coronavirus infection (e.g., COVID-19 infection). In certain embodiments, the present disclosure provides a pharmaceutical composition comprising an inhibitor of S-adenosylhomocysteine (SAH) hydrolase. In some embodiments, the inhibitor of SAH hydrolase is a compound selected from Table 1. In some embodiments, the inhibitor of SAH hydrolase is a compound of Formula I or Formula II or a pharmaceutically acceptable salt thereof.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, l-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, the pharmaceutical composition comprises about 10 mg to 100 mg, about 10 mg to 80 mg, about 10 mg to 60 mg, about 10 mg to 40 mg, or about 20 mg to 40 mg of the compound of Table 1, Formula I, or Formula II. In certain embodiments, the pharmaceutical composition comprises about 10 mg, about 15 mg, about 20 mg, about 25 mg, about 30 mg, about 35 mg, about 40 mg, about 45 mg, or about 50 mg of the compound of Table 1, Formula I, or Formula II. In preferred embodiments, the pharmaceutical composition comprises about 30 mg of the compound of Table 1, Formula I, or Formula II.

In some embodiments, the pharmaceutical composition is an inhalation composition. In some embodiments, the pharmaceutical composition is a dry powder inhalation composition. In some embodiments, the pharmaceutical composition is an injectable formulation. In some embodiments, the pharmaceutical composition is an intravenous infusion formulation. In some embodiments, the pharmaceutical composition is a subcutaneously injectable formulation.

### Methods of Use

In certain aspects, provided herein are methods of treating a coronavirus infection (e.g., COVID-19) comprising administering to a patient in need thereof an inhibitor of SAH hydrolase. In some embodiments, the inhibitor of SAH hydrolase is a compound selected from Table 1. In some embodiments, the inhibitor of SAH hydrolase is a compound of Formula I or Formula II or a pharmaceutically acceptable salt thereof.

In certain other aspects, provided herein are methods of treating a coronavirus infection (e.g., COVID-19), comprising administering a pharmaceutical composition disclosed herein.

In certain embodiments, the inhibitor of SAH hydrolase, or pharmaceutical composition is administered intravenously, intrathecally, subcutaneously, intramuscularly, intranasally, or orally.

In some embodiments, the pharmaceutical composition is administered for a treatment period from 1 day to 42 days. In some embodiments, the pharmaceutical composition is administered for a treatment period from 21 days to 35 days. In some embodiments, the pharmaceutical composition is administered for a treatment period from 3 days to 10 days. In some embodiments, the pharmaceutical composition is administered for a treatment period for about 7 days. In some embodiments, the pharmaceutical composition is administered for a treatment period of about 28 days.

The effective amount of a compound of the invention in such a therapeutic method is from about 0.01 mg/kg/day to about 1000 mg/kg/day, from about 0.1 mg/kg/day to about 100 mg/kg/day, from about 0.5 mg/kg/day to about 50 mg/kg/day, or from about 1 mg/kg/day to 10 mg/kg/day. In some embodiments, the effective amount of a compound of the invention in such a therapeutic method is about 2 mg/kg/day, about 5 mg/kg/day, about 7.5 mg/kg/day, about 10 mg/kg/day, about 12.5 mg/kg/day, about 15 mg/kg/day, or about 20 mg/kg/day.

In some embodiments, the effective amount of the compound of Formula I or Formula II is about 10 mg to 100 mg, about 10 mg to 80 mg, about 10 mg to 60 mg, about 10 mg to 40 mg, or about 20 mg to 40 mg. In certain embodiments, the effective amount of the compound of Formula I or Formula II is about 10 mg, about 15 mg, about 20 mg, about 25 mg, about 30 mg, about 35 mg, about 40 mg, about 45 mg, or about 50 mg of the compound of Formula I or Formula II. In preferred embodiments, the effective amount of the compound of Formula I or Formula II is about 30 mg.

In some embodiments, the effective amount of the inhibitor of SAH hydrolase is about 10 mg to 100 mg, about 10 mg to 80 mg, about 10 mg to 60 mg, about 20 mg to 80 mg, or about 30 mg to 60 mg. In certain embodiments, the effective amount of the inhibitor of SAH hydrolase is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg. In preferred embodiments, the effective amount of the inhibitor of SAH hydrolase is about 50 mg.

In some embodiments, the pharmaceutical composition is administered from 1 to 5 times a day. In some embodiments, the pharmaceutical composition is administered 3 times a day. In some embodiments, the pharmaceutical composition is administered with a frequency from about every 2 hours to about every 6 hours. In some embodiments, the pharmaceutical composition is administered with a frequency of about every 4 hours.

In some embodiments, the method comprises administering the pharmaceutical composition by intravenous infusion, by intravenous injection, by subcutaneous injection, by intramuscular injection, by intraperitoneal injection, orally, sublingually, buccally, or by inhalation. In some embodiments, the method comprises administering the pharmaceutical composition by intravenous infusion or by intravenous injection. In some embodiments, the method comprises administering the pharmaceutical composition orally. In some preferred embodiments, the method comprises administering the pharmaceutical composition by inhalation. In some embodiments, the method comprises administering the pharmaceutical by inhalation using a nebulizer.

In some embodiments, the coronavirus is selected from SARS-CoV, MERS-CoV, HCoV, HKU1, and SARS-CoV-2. In some embodiments, the coronavirus is selected from SARS-CoV, MERS-CoV, and SARS-CoV-2. In preferred embodiments, the coronavirus is SARS-CoV-2.

In some embodiments, the subject has SARS-CoV-2 infection, e.g., as confirmed by reverse-transcription polymerase chain reaction (RT-PCR) from respiratory tract or blood specimens.

### EXAMPLES

The invention now being generally described, it will be more readily understood by reference to the following examples, which are included merely for purposes of illustration of certain aspects and embodiments of the present invention, and are not intended to limit the invention.

### Example 1: In Vitro Assay System for Viral Growth and Cytotoxicity Assays

Compounds of the invention may be prepared according to the synthetic procedures described in scheme 1. In cases where the synthetic intermediates and final products of Formula I described below contain potentially reactive functional groups, for example amino, hydroxy, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999).

Compounds of the invention can be prepared employing conventional methods that utilize readily available reagents and starting materials. The reagents used in the preparation of the compounds of this invention can be either commercially obtained or can be prepared by standard procedures described in the literature. The compounds of the invention may be made according to the general and exemplary schemes provided herein.

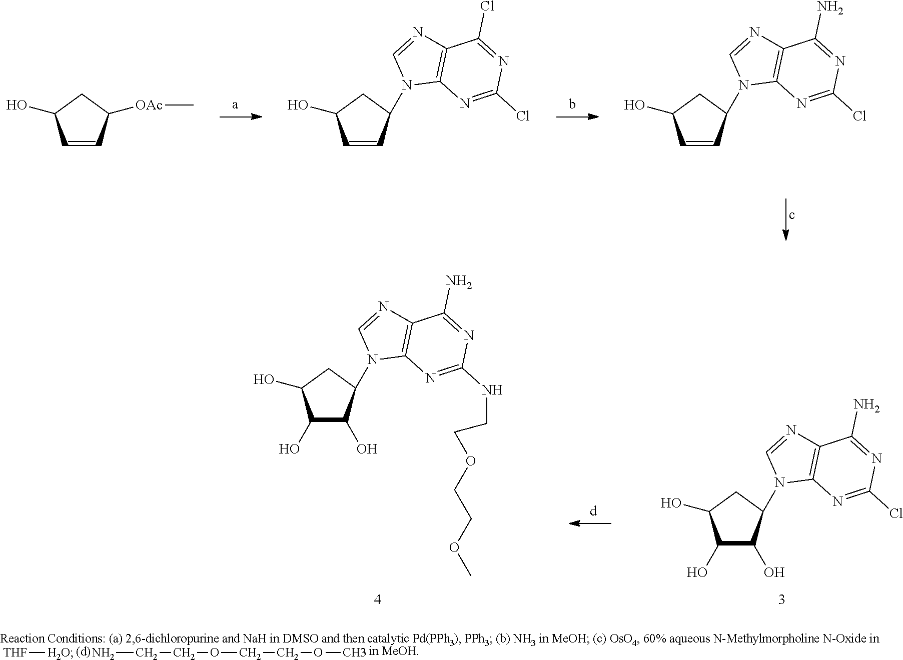

For example (1S,2R,3S,4R)-4-[6-Amino-2-[2-[2-(2-Aminoethoxy)ethoxy]ethanol]-9H-purin-9-yl]cyclopentane-1,2,3-triol (4) was synthesized

A suspension of compound 3 (300 mg, 1.05 mmol) (See S. Siddiqi, et. al. J Med Chem. 1995 Mar. 31; 38(7): 1174-1188) in absolute EtOH (10 mL) was added to 2-[2-(2-Aminoethoxy)ethoxy]ethanol (300 mg, 2.01 mmol). This mixture was heated at 90° C. for 3 days. The solvent was removed by rotary evaporation, and the residue was subjected to column chromatography on silica gel (eluent CH2Cl2-MeOH) to result in compound 4 (272 mg, 70%) as a yellow solid: mp 200° C.

### Example 2: In Vitro Assay System for Viral Growth and Cytotoxicity Assays

A new class of broad-spectrum antiviral drugs is developed to address the current COVID-19 pandemic and any other potential viral epidemic. The parent molecule is tested in in vitro assay system for viral growth and cytotoxicity assays. A series of its derivatives are synthesized and tested to develop Structure Activity Relationship (SAR) with SARS-CoV-2 and SAH hydrolase inhibition to select the best candidates.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of treating coronavirus infection in a subject, comprising administering to the subject a compound of Formula I or Formula II: 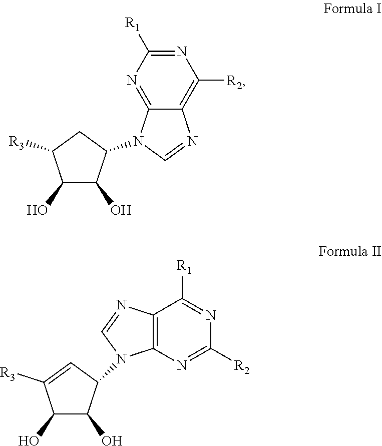
or a pharmaceutically acceptable salt thereof;
wherein
R₁ is H, halogen, NH₂, OH, SH, NH-Allyl, NH-alkyl, NH-aryl, NH-alkynyl, NH—CH₂—CH₂—O—CH₂—CH₂—O—CH₃, NH-PEG, O-alkyl, O-aryl, O-alkynyl, O-PEG, S-alkyl, S-aryl, S-alkynyl, S-PEG, alkyl, alkynyl, aryl, heteroaryl, and heterocyclyl;
R₂ is selected from H, halogen, NH₂, OH, SH, NH-Allyl, NH-alkyl, NH-aryl, NH-alkynyl, NH—CH₂—CH₂—O—CH₂—CH₂—O—CH₃, NH-PEG, O-alkyl, O-aryl, O-alkynyl, O-PEG, S-alkyl, S-aryl, S-alkynyl, S-PEG, alkyl, alkynyl, aryl, heteroaryl, and heterocyclyl; and
R₃ is selected from —H, —OH, -alkyl-OH, -alkylene-OH, alkyl, alkynyl, aryl, heteroaryl, and heterocyclyl.

2. The method of claim 1, wherein the compound of Formula I is: 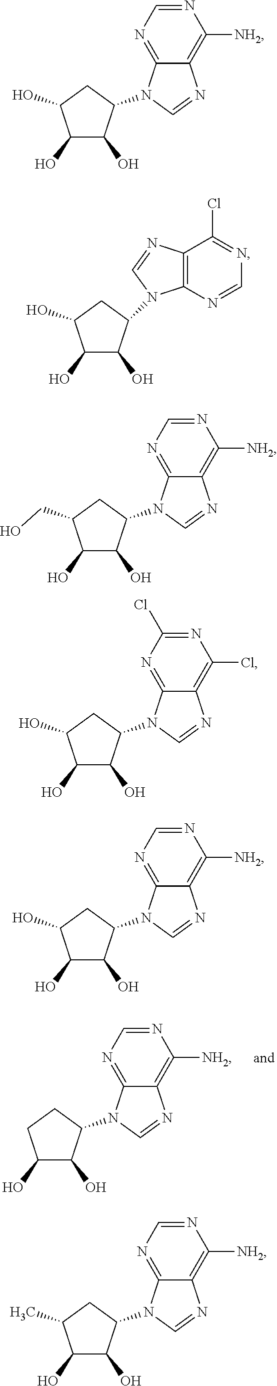
or a pharmaceutically acceptable salt thereof.

3. The method of claim 1 or 2, wherein the compound of Formula II is: 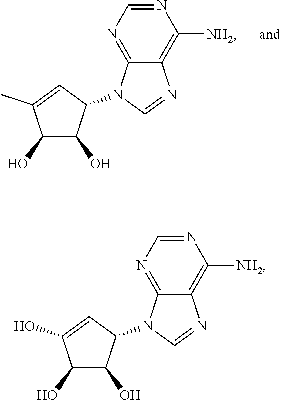
or a pharmaceutically acceptable salt thereof.

4. The method of any one of claims 1-3, wherein the compound is formulated for oral, rectal, intravenous, or subcutaneous formulation.

5. The method of any one of the claims 1-3, wherein the compound is formulated for inhalation.

6. The method of claim 5, wherein the compound is formulated as a dry powder.

7. The method of any one of claims 1-6, wherein the coronavirus is selected from SARS-CoV, MERS-CoV, HCoV, HKU1, and SARS-CoV-2.

8. The method of claim 7, wherein the coronavirus is SARS-CoV-2.

9. The method of any one of claims 1-8, wherein the subject has SARS-CoV-2 infection, which has been confirmed by reverse-transcription polymerase chain reaction (RT-PCR) from respiratory tract or blood specimens.

10. A method of treating coronavirus infection in a subject, comprising administering to the subject an inhibitor of S-adenosylhomocysteine (SAH) hydrolase.

11. The method of claim 10, wherein the inhibitor of SAH hydrolase is a compound selected from Table 1.

12. The method of any one of claims 10-11, wherein the inhibitor of SAH hydrolase is formulated for oral, rectal, intravenous, or subcutaneous formulation.

13. The composition of any one of the claims 10-12, wherein the inhibitor of SAH hydrolase is formulated for inhalation.

14. The composition of claim 13, wherein the inhibitor of SAH hydrolase is a dry powder for inhalation.

15. The method of any one of claims 10-14, wherein the coronavirus is selected from SARS-CoV, MERS-CoV, HCoV, HKU1, and SARS-CoV-2.

16. The method of claim 15, wherein the coronavirus is SARS-CoV-2.

17. The method of any one of claims 10-16, wherein the subject has SARS-CoV-2 infection, such as an infection confirmed by reverse-transcription polymerase chain reaction (RT-PCR) from respiratory tract or blood specimens.

18. A pharmaceutical composition comprising a compound of Formula I or Formula II: 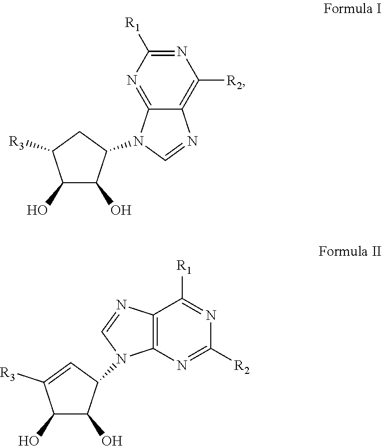
or a pharmaceutically acceptable salt thereof;
wherein
R₁ is selected from H, halogen, NH₂, OH, SH, NH-Allyl, NH-alkyl, NH-aryl, NH-alkynyl, NH—CH₂—CH₂—O—CH₂—CH₂—O—CH₃, NH-PEG, O-alkyl, O-aryl, O-alkynyl, O-PEG, S-alkyl, S-aryl, S-alkynyl, S-PEG, alkyl, alkynyl, aryl, heteroaryl, and heterocyclyl;
R₂ is selected from H, halogen, NH₂, OH, SH, NH-Allyl, NH-alkyl, NH-aryl, NH-alkynyl, NH—CH₂—CH₂—O—CH₂—CH₂—O—CH₃, NH-PEG, O-alkyl, O-aryl, O-alkynyl, O-PEG, S-alkyl, S-aryl, S-alkynyl, S-PEG, alkyl, alkynyl, aryl, heteroaryl, and heterocyclyl;
R₃ is selected from —H, —OH, -alkyl-OH, -alkylene-OH, alkyl, alkynyl, aryl, heteroaryl, and heterocyclyl; and
a pharmaceutically acceptable excipient.

19. The composition of claim 18, wherein the compound of Formula I is selected from: 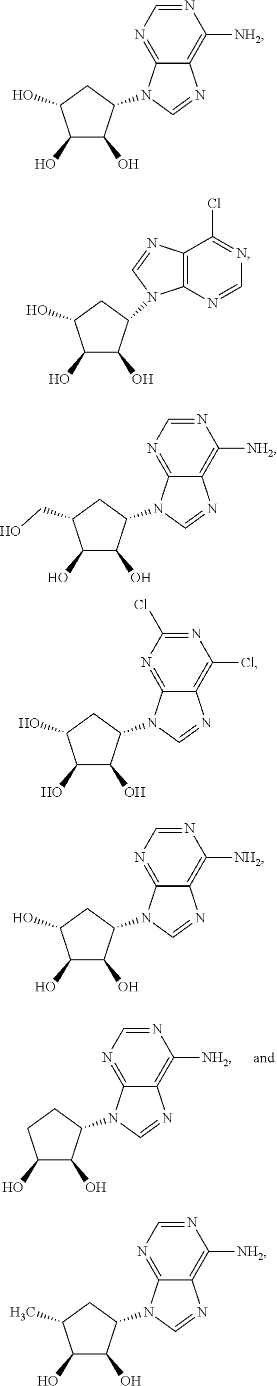
or a pharmaceutically acceptable salt thereof.

20. The composition of claim 18 or 19, wherein the compound of Formula II is selected from: 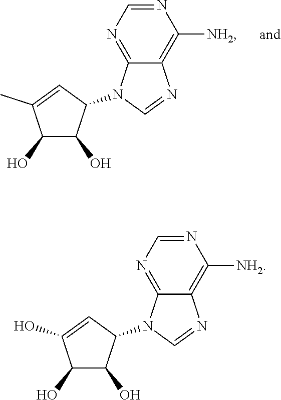
or a pharmaceutically acceptable salt thereof.

21. The composition of any one of claims 18-20, wherein the composition is formulated for oral, rectal, intravenous, or subcutaneous formulation.

22. The composition of any one of the claims 18-20, wherein the composition is formulated for inhalation.

23. The composition of claim 22, wherein the composition is formulated as a dry powder.

24. An inhaler comprising the composition of any one of claims 18-23.

25. An inhalation composition, wherein the inhalation composition comprising the composition of any one of claims 18-23.

26. An inhaler comprising the inhalation composition of claim 25.
